(19) United States
(12) Patent Application Publication
(10) Pub. No.: US 2023/0310465 A1
(43) Pub. Date: Oct. 5, 2023

(54) NANO LIPID CARRIER SYSTEM FOR IMPROVING PERMEATION OF ACTIVE INGREDIENTS

(71) Applicant: LEIUTIS PHARMACEUTICALS LLP, Hyderabad (IN)

(72) Inventors: Chandrashekhar KOCHERLAKOTA, Hyderabad (IN); Nagaraju BANDA, Hyderabad (IN); Arjun NARALA, Hyderabad (IN); Naga Udaya Sankar PULLAGURA, Hyderabad (IN); Srinath AKULA, Hyderabad (IN)

(21) Appl. No.: 18/021,647

(22) PCT Filed: Aug. 18, 2021
(86) PCT No.: PCT/IB2021/057584

§ 371 Date: Feb. 16, 2023

(30) Foreign Application Priority Data
Aug. 18, 2020 (IN) 202041035519

(51) Int. Cl. A61K 31/00; A61K 9/127; A61K 9/00; A61K 9/51; A61K 47/44

(52) U.S. Cl. CPC A61K 31/658; A61K 9/1271; A61K 9/0053; A61K 9/5192; A61K 47/44

(57) ABSTRACT

The present invention relates to a formulation comprising a nano lipid carrier system comprising an active ingredient, low phase transition solid lipid, PEGylated lipid, surfactants and cosolvents etc, dispersed in water or aqueous medium. The hydrodynamic diameter of the nanocarrier system in aqueous media is less than about 1000 nm. The present invention formulation can be in the form of a liquid (solution, suspension, dispersion), liquid filled hard gelatin capsule, soft gelatin capsule or a liquid concentrate.

### FIELD OF THE INVENTION

This application is a National Stage Application under 35 U.S.C. § 371 of PCT International Application No. PCT/IB2021/057584, filed Aug. 18, 2021, which takes priority from Indian Application Number 202041035519, filed Aug. 18, 2020, all of which are herein incorporated by reference in their entireties.

The present invention relates to a formulation comprising a nano lipid carrier system comprising an active ingredient, low phase transition substance, PEGylated lipid, surfactants and co-solvents, dispersed in water or aqueous medium. The hydrodynamic diameter of the nanocarrier system in aqueous media is less than about 1000 nm. The present invention formulation can be in the form of a liquid (solution, suspension, and dispersion), liquid filled hard gelatin capsule, soft gelatin capsule or a liquid concentrate.

### BACKGROUND OF THE INVENTION

Nanotechnology has been widely applied in pharmaceuticals for drug delivery. The technology has proven to be useful in formulating delivery systems for small molecules, proteins etc. These improved drug delivery systems can address issues associated with currently used drugs such as increasing efficacy or improving safety and patient compliance. Therefore, nanoparticle systems, such as liposomes, nanoemulsions, and polymeric nanoparticles, have gradually emerged as most investigated systems for drug delivery.

Many therapeutic agents have not been successful because of their limited ability to reach the target tissue. Further, the conventional delivery systems present a lot of disadvantages with regards to safety and efficacy; administration modalities; patient compliance etc.

The advantages of using nanoparticles for drug delivery result from their two main properties. First, nanoparticles, because of their small size, can penetrate through smaller capillaries and are taken up by cells, which allows efficient drug accumulation at the target sites. Second, the use of biodegradable materials for nanoparticle preparation allows window to control the drug release with minimum side effects.

U.S. Pat. No. 4,107,288 describes particles in the size range from 10 to 1,000 nm containing a biologically or pharmacodynamically active material. However, the particles comprise a crosslinked matrix of macromolecules having the active material supported on or incorporated into the matrix.

U.S. Pat. No. 5,145,684 describes a method for providing drug particles having an effective average particle size of less than about 400 nm. The method includes wet milling the drug in the presence of a grinding medium in conjunction with a surface modifier. As in previous methods, the '684 protocol requires grinding or milling to achieve size reduction. The method further requires the use of an additive in the form of a surface modifier.

It is desirable to provide stable dispersible drug particles in nano size range that can be readily prepared using simple and reproducible manufacturing techniques. Moreover, it would be highly desirable to provide pharmaceutical compositions having enhanced permeability.

### SUMMARY OF THE INVENTION

One aspect of the invention is to provide a nano lipid carrier system with one or more low phase transition substance selected from the group comprising a low phase transition lipid, cannabidiol or mixtures thereof, oil, PEGylated lipid, surfactant and a co-solvent.

Another aspect of the invention is to provide the said nano lipid carrier system wherein the hydrodynamic diameter is less than about 1000 nm.

Another aspect of the invention is to provide a nano lipid carrier system comprising cannabidiol, one or more active ingredients, oil, PEGylated lipid, surfactant and a co-solvent.

### BRIEF DESCRIPTION OF THE DRAWINGS

FIG. 1 shows comparative permeability flux of netupitant from formulations evaluated using rat intestine (mcg/cm²/h).

FIG. 2 shows comparative permeability flux of netupitant and CBD from formulations evaluated using rat intestine (mcg/cm²/h).

FIG. 3 shows comparative Permeability flux of Netupitant from various NLC formulations evaluated using Rat Intestine (mcg/cm²/h).

### DETAILED DESCRIPTION OF THE INVENTION

The present invention relates to a novel nano lipid carrier system that improves the oral permeation of active ingredient. The inventors formulated a novel nano lipid carrier (NLC) system that showed unexpected results in improving permeation rate of active ingredient by more than about five fold.

The NLC system of the present invention comprises (i) one or more low phase transition substance, (ii) oil, (iii) PEGylated lipid, (iv) surfactant and (v) a co-solvent. The low phase transition substance may be selected from the group comprising a low phase transition lipid, cannabidiol or mixtures thereof. The inventors surprisingly found that the presence of PEGylated lipid in the NLC formulation increased the permeability of cannabidiol by two to four times.

The NLC system of the present invention has a hydrodynamic diameter (HDD) of less than about 1000 nm in aqueous media. Specifically, the system has a hydrodynamic diameter less than about 700 nm. More specifically, the hydrodynamic diameter (HDD) of the present invention for NLC formulations have greater than 90% of NLC particles in the range of 100-250 nm and for self-dispersible NLC not less than 70% of self-dispersible NLC particles fall in the range of 250-500 nm. The hydrodynamic diameter can be measured using Particle Size Analyzer (Lite Sizer, Anton Paar). Any of the well-known techniques such as high shear and high pressure homogenization, microfluidization, and membrane emulsification techniques can be used for making the said carrier system.

The novel Nano lipid carrier system can also be formulated as a self-dispersible concentrate comprising of: low phase transition (LPT) substance selected from the group comprising a low phase transition lipid, cannabidiol or mixtures thereof, oil, PEGylated lipid. The system may additionally contain co-solvents, surfactants, stabilizers and optionally organoleptic modifiers. The formulation may be presented as self-dispersible concentrate in liquid or capsule dosage form or as concentrate adsorbed onto a powder matrix. The self-dispersible concentrate when added to aqueous medium forms nanodispersion with minimal agitation.

The NLC system of the present invention comprises
i. one or more low phase transition substance selected from the group comprising a low phase transition lipid, cannabidiol or mixtures thereof,
ii. oil,
iii. PEGylated lipid
iv. surfactant and
v. a co-solvent.

The phase transition temperature herein is defined as the temperature required to induce a change in the lipid physical state from the ordered phase, where the hydrocarbon chains are fully extended and closely packed, to the disordered phase, where the hydrocarbon chains are randomly oriented and fluid. The low phase transition lipid used in this invention is a substance that has a phase transition temperature below 80° C. It may be selected from the group comprising decanoic acid, propyl glycol monostearate (Monosteol), alpha-lipoic acid (ALA), cholesterol, triacylglycerols like tricaprin, trilaurin, trimyristin, tripalmitin and tristearin, acylglycerols like glycerol monostearate (Imwittor 900), glycerol behenate (Compritol 888 ATO) and glycerol palmitostearate (Precirol ATO 5), fatty acids like stearic acid, palmitic acid and myristic acid, lauric acid, waxes like carnauba wax, cetyl palmitate and the like. The lipid may not be limited to the excipients described herein and may be a mixture of one or more lipids. Decanoic acid, monosteol, alpha lipoic acid, cholesterol are preferred. The low phase transition substance may also be cannabidiol and its derivatives.

Cannabidiol has the following structure and is chemically 2-[(1R, 6R)-3-Methyl-6-(1-methylethenyl)-2cyclohexen-1-yl]-5-pentyl-1,3-benzenediol. Its empirical formula is C₂₁H₃₀O₂.

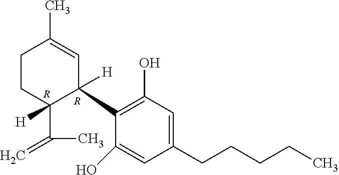

Cannabidiol is approved in the US as Epidiolex for the treatment of seizures associated with Lennox-Gastaut syndrome, Dravet syndrome, or Tuberous sclerosis complex in patients 1 year of age and older.

TABLE 1
List of low phase transition lipids

| | | Transition | |
|---|---|---|---|
| S. No | Name of Lipids | temperature | |
| 1. | decanoic acid | 31.2-33.2° | C. |
| 2. | alpha lipoic acid | 60-62° | C. |
| 3. | Monosteol (propylene glycol monostearate) | 35-38.5° | C. |
| 4. | Precirol ATO 5 (glyceryl palmitostearate) | 50-60° | C. |
| 5. | Imwittor 900 (glycerol monostearate) | 55-60° | C. |
| 6. | Compritol 888 ATO (glycerol behenate) | 65-77° | C. |
| 7. | lauric acid | 44-46° | C. |
| 8. | myristic acid | 52-54° | C. |
| 9. | stearic acid | 67-72° | C. |
| 10. | palmitic acid | 61-62.5° | C. |
| 11. | Dynasan 114 (glyceryl trimyristate) | 56-57° | C. |
| 12. | Dynasan 116 (glyceryl tripalmitate) | 66-67° | C. |
| 13. | Gelucire 50/13 (stearoyl polyoxylglycerides) | 46-51° | C. |

If the low phase transition substance is a lipid, it may be present up to 20%, more preferably up to 15% by weight of the composition. If the low phase transition substance selected is cannabidiol, it may be present up to 35%, preferably up to 30% by weight of the composition.

The oil can be selected from the group comprising medium chain triglycerides such as Labrafac Liphophile WL 1349 (Medium chain Triglycerides NF), caprylic acid and the like, fish Oil (Incromega), soyabean oil, olive oil, corn oil, vitamin E, grapeseed oil, walnut oil, avocado oil, flax seed oil, coconut oil, olive oil, hemp seed oil, ginger oil, eicosapentaenoic acid (EPA), docosahexaenoic acid (DHA), omega fatty acid (OFA) and the like. Medium chain triglycerides, fish Oil, omega fatty acid are preferred. The oil may constitute up to 50% by weight of the composition or more preferably up to 45% by weight of the composition.

PEGylated lipid herein shall mean a Lipid molecule with attached PEG wherein molecular weight of PEG ranges from 200 Da to 20000 Da. The class of lipids encompasses fatty acids, sterols, glycerol derivatives of fatty acids, monoglycerides, diglycerides, triglycerides, hydrophobic or amphiphilic molecules, waxes, phospholipids, sterols, vitamins, oils, cholesterol, and of the kind. More specifically, the PEGylated lipids can be chosen from the group comprising N-(Carbonyl-methoxypolyethylenglycol-2000)-1,2-distearoyl-sn-glycero-3-phosphoethanolamine, MPEG-2000-DSPE, Na-salt (LIPOID PE 18:0/18:0-PEG 2000), Polyethylene glycol-15-hydroxystearate (Solutol HS-15), N-(Carbonyl-methoxypolyethylenglycol-2000)-1,2-dimyristoyl-sn-glycero-3 phosphoethanolamine, MPEG-2000-DMPE, sodium salt (LIPOID PE 14:0/14:0- PEG 2000), N-(Carbonyl-methoxypolyethylenglycol-5000)-1,2-dimyristoyl-sn-glycero-3-phosphoethanolamine, MPEG-5000-DMPE, sodium salt (LIPOID PE 14:0/14:0-PEG 5000), N-(Carbonyl-methoxypolyethylenglycol-2000)-1,2-dipalmitoyl-sn-glycero-3-phosphoethanolamine, MPEG-2000-DPPE, sodium salt.

(LIPOID PE 16:0/16:0-PEG 2000), N-(Carbonyl-methoxy polyethylene glycol-5000)-1,2-dipalmitoyl-sn-glycero-3-phosphoethanolamine, MPEG-5000-DPPE, sodium salt (LIPOID PE 16:0/16:0-PEG 5000), N-Carbonyl methoxy polyethylene glycol-5000)-1,2-distearoyl-sn-glycero-3-phospho ethanolamine, MPEG-2000-DSPE, Na-salt (LIPOID PE 18:0/18:0-PEG 5000) (PEG-PE), Polyethylene glycol monostearate (Gelucire® 48/16).

The most preferred PEGylated lipid is PEG-PE. The inventors surprisingly found that permeability of cannabidiol (CBD) is increased in the NLC system by about 2 fold in presence of PEGylated lipid than compared to corresponding preparation without PEGylated lipid. This data is presented in table 6. Further, the inventors also surprisingly found that permeability of active ingredient increased by about 4 times in NLC that contained combination of cannabidiol and PEGylated lipid than compared to NLC formulation of active ingredient that did not contain cannabidiol and PEGylated lipid. The PEGylated lipids, preferably PEG-PE, may be present up to 10% by weight of the composition.

The co solvents can be selected from the group comprising ethanol, N-methyl-2-pyrrolidone (NMP), tertiary butyl alcohol (TBA), glycerol, propylene glycol, polyethylene glycol, Transcutol HP, Gelucire® 50/13 (stearoyl polyoxylglycerides). The cosolvents may be present up to 10% by weight of the composition.

Surfactants can be selected from the group comprising amphiphilic surfactants, lipophilic surfactants comprising like alpha lecithin, cholesterol oleate; phospholipids like soy lecithin, egg lecithin (LIPOID E 80 S), hydrogenated soy lecithin, alpha lecithin; modified phospholipids derivatives of dimyristoyl phosphatidylcholine (DMPC), 1,2-dipalmitoyl-rac-glycero-3-phosphocholine (DPPC), dimyristoyl phosphatidyl ethanolamine (DMPE), 1,2-bis(diphenylphosphino) ethane (DPPE), dimyristoylphosphatidylglycerol (DMPG), dipalmitoyl phosphatidylglycerol (DPPG), 1,2-distearoyl-sn-glycero-3-hosphorylethanolamine (DSPE), 1,2-dioleoyl-sn-glycero-3-phosphoethanolamine (DOPE), hydrophilic surfactants like poloxamer-188, sodium oleate, sodium cholate, ethylene oxide/propylene oxide copolymers like poloxamer 182, poloxamer 407 and poloxamine 908, poloxamer-188, non-ionic surfactants like polysorbate 20, polysorbate 60, polysorbate 80 (Tween 80), Span-20, Span-60 and Span-80, cremophor EL, anionic bile salts sodium cholate, sodium glycocholate, sodium tauro cholate, sodium tauro deoxy cholate and sucrose esters of fatty acids (ex: sucrose laurate, sucrose oleate, sucrose palmitate, sucrose stearate and the like), Kolliphor RH40, as aqueous solution or dispersion of surfactants. The quantity of surfactants may be up to 10% by weight of the composition.

The stabilizers may be selected from antioxidants like vitamin E acetate, ascorbyl palmitate, ascorbic acid, monothioglycerol (MTG), citric acid, tartaric acid etc, chelating agents like 1,4,7,10-tetraazacyclododecane-1,4,7,10-tetraacetic acid (DOTA), disodium EDTA, preservatives like methyl paraben, propyl paraben, chlorbutanol and the like.

Buffers may be selected from the group comprising L-histidine, L-arginine, tris base, meglumine, glycine, sodium succinate, diethanol amine, aspartic acid, glutamic acid, alanine, sodium acetate, sodium ascorbate, sodium citrate, citric acid, succinic acid, triethanolamine, boric acid, sodium hydroxide, sodium bicarbonate etc. L-histidine, L-arginine, tris base, meglumine, glycine are preferred.

The NLC system may also contain tonicity modifiers like dextrose, mannitol, glycerol, sodium chloride, organoleptic additives like sucralose, aspartame, saccharin, neohesperidine dihydrochalcone, neotame, alitame, erythritol flavouring agents like peppermint supreme, masking agent 2522, pineapple flavour, lemon flavour, lime flavor, masking agent 2521, menthol, spearmint oil, cinnamon oil.

In another embodiment, the NLC system is used to formulate a combination of cannabidiol with netupitant, palonosetron, atorvastatin, gabapentin, naproxen, abiraterone, cabazitaxel, paclitaxel, duloxetine and many other active ingredients and combinations thereof.

Netupitant is 2-[3,5-bis(trifluoromethyl)phenyl]-N,2-dimethyl-N-[4-(2methylphenyl)-6-(4-methylpiperazin-1-yl)pyridin-3-yl]propenamide. Netupitant in combination with Palonosetron is commercially marketed as Akynezo as a capsule. The absorption of Netupitant from Akynezo is reported to be in range of 63% to 87% with Tmax of about 5hrs in healthy subjects. Netupitant has the following chemical structure:

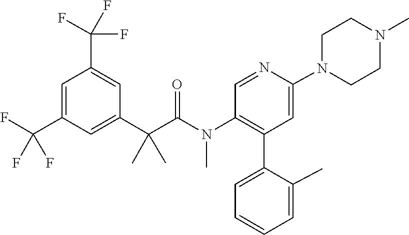 [SMILES: Cc1ccccc1-c1cc(N2CCN(C)CC2)ncc1N(C)C(=O)C(C)(C)c1cc(C(F)(F)F)cc(C(F)(F)F)c1]

It is used for the prevention of acute and delayed chemotherapy-induced nausea and vomiting, including highly emetogenic chemotherapy such as with cisplatin.

Palonosetron hydrochloride is chemically (3aS)-2-[(S)-1-Azabicyclo [2.2.2]oct-3-yl]-2,3,3a,4,5,6-hexahydro-1-oxo-1Hbenz[de]isoquinoline hydrochloride. The empirical formula is C19H24N2O.HCl, with a molecular weight of 332.87. Palonosetron hydrochloride exists as a single isomer and has the following structural formula:

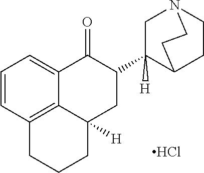 [SMILES: O=C1c2cccc3c2[C@H](CCC3)CC1[C@@H]1CN2CCC1CC2 ; Cl]

Palonosteron hydrochloride is marketed as Aloxi injection and capsules and is indicated for the treatment of acute and delayed nausea and vomiting associated with initial and repeat courses of moderately emetogenic cancer chemotherapy (MEC); acute nausea and vomiting associated with initial and repeat courses of highly emetogenic cancer chemotherapy (HEC); postoperative nausea and vomiting (PONV) and acute nausea and vomiting associated with initial and repeat courses of emetogenic cancer chemotherapy, including highly emetogenic cancer chemotherapy (HEC).

The formulation of the present invention can be presented in any of the following dosage forms:
i. liquid (solution or suspension)
ii. soft gelatin capsule
iii. liquid filled hard gelatin capsule
iv. a kit with a liquid concentrate and a diluent
v. a liquid (solution or suspension) that is diluted with water or any other suitable diluent before administration

It is to be understood that these active ingredients are chosen for demonstrating the efficacy of the system and are not be considered as being limited to the said examples.

### Examples

### Nano Lipid Carrier System Compositions

### Example 1: Compositions of Nano Lipid Carrier System NLC

| | | Quantity |
|---|---|---|
| S. No | Ingredients | (% w/v) |
| 1. | decanoic acid | 0.8-1.2 |
| 2. | Labrafac Lipophile WL 1349 (Medium | 0.6-1.2 |
| | Chain Triglycerides NF) | |
| 3 | N-(Carbonyl methoxypolyethylenglycol-2000)- | 0.3-1.2 |
| | 1,2-distearoyl-sn-glycero-3- | |
| | phosphoethanolamine, MPEG-2000-DSPE, | |
| | Na-salt (Lipoid PE 18:0/18:0-PEG 2000) | |
| 4. | ethanol | 0.5-1.0 |
| 5 | egg lecithin (Lipoid E 80S) | 0.5-1.0 |
| 6. | cholesterol | 0.2-0.3 |
| 7. | poloxamer-188 | 0.5-1.0 |
| 8. | vitamin E acetate | 0.01-0.02 |
| 9 | 1,4,7,10-Tetraazacyclododecane- | 0.001-0.002 |
| | 1,4,7,10-Tetraacetic acid (DOTA) | |
| 10. | propyl paraben | 0.01-0.02 |
| 11. | chlorbutanol | 0.01-0.025 |
| 12. | tris base | 0.1-0.15 |
| 13. | dextrose anhydrous | 3.5-4.0 |
| 14. | sucralose | 0.05-0.1 |
| 15. | peppermint supreme | 0.05-0.1 |
| 16. | Purified Water | Q.S to 100% |

Preparation of Oil Phase: Accurately weighed quantity of vitamin E acetate was taken into a manufacturing vessel. Labrafac Lipophile WL 1349 (oil) was subsequently added to it followed by decanoic acid and dissolved by heating on a water bath at 55° C. (50° C. to 60° C.). Weighed quantities of cholesterol, PEG-PE and egg lecithin (Lipoid E 80S) were further added and dissolved. This mixture was cooled to 40° C., ethanol, propyl paraben and peppermint supreme flavor were added.

Preparation of Aqueous Phase: 90% of required quantity of Milli-Q water per batch was taken in another manufacturing vessel in which weighed quantity of poloxamer-188 was added and dissolved by heating at 55° C. (50° C. to 60° C.). Dextrose, sucralose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The Size and Zeta Potential of the undiluted NLC dispersion were measured using Particle Size Analyzer (Lite Sizer, Anton Paar).

### Example 2: Oral Nanocarrier Formulation of Netupitant and Palonosetron with Various PEGylated Lipid Concentrations

| | | Batch# | | |
|---|---|---|---|---|
| | | 135A | 135C | 135D |
| | | (0% | (0.6% | (1.2% |
| | | PEGylated | PEGylated | PEGylated |
| | | lipid) | lipid) | lipid) |
| S. No | Ingredients | %(w/v) | | |
| 1 | decanoic acid | 1.200 | 1.200 | 1.200 |
| 2 | Labrafac Lipophile WL | 1.200 | 1.200 | 1.200 |
| | 1349 (Medium Chain | | | |
| | Triglycerides NF) | | | |
| 3 | N-(Carbonyl- | 0.000 | 0.600 | 1.200 |
| | methoxypolyethylenglycol- | | | |
| | 2000)-1,2-distearoyl-sn- | | | |
| | glycero-3- | | | |
| | phosphoethanolamine, | | | |
| | MPEG-2000-DSPE, Na- | | | |
| | salt (Lipoid PE 18:0/18:0- | | | |
| | PEG 2000) | | | |
| 4 | netupitant | 0.600 | 0.600 | 0.600 |
| 5 | palonosetron hydrochloride | 0.001 | 0.001 | 0.001 |
| 6 | ethanol | 1.000 | 1.000 | 1.000 |
| 7 | soy lecithin (Lipoid S 100) | 0.500 | 0.500 | 0.500 |
| 8 | cholesterol | 0.300 | 0.300 | 0.300 |
| 9 | poloxamer-188 | 1.000 | 1.000 | 1.000 |
| 10 | vitamin E acetate | 0.020 | 0.020 | 0.020 |
| 11 | propyl paraben | 0.020 | 0.020 | 0.020 |
| 12 | 1,4,7,10- | 0.002 | 0.002 | 0.002 |
| | Tetraazacyclododecane- | | | |
| | 1,4,7,10-tetraacetic acid | | | |
| | (DOTA) | | | |
| 13 | dextrose anhydrous | 3.500 | 3.500 | 3.500 |
| 14 | sucralose | 0.100 | 0.100 | 0.100 |
| 15 | peppermint supreme | 0.100 | 0.100 | 0.100 |
| 16 | tris base | 0.100 | 0.100 | 0.100 |
| 17 | Milli-Q Water | Q.S to | Q.S to | Q.S to |
| | | 100% | 100% | 100% |

### Manufacturing Procedure

Preparation of Oil Phase: Accurately weighed quantity of vitamin E acetate was taken into a manufacturing vessel. Labrafac Lipophile WL 1349 (oil) was subsequently added to it followed by decanoic acid and dissolved by heating on a water bath at 55° C. (50° C. to 60° C.). Weighed quantities of cholesterol, PEG-PE and Soy lecithin (Lipoid S 100) were further added and dissolved. This mixture was cooled to 40° C., ethanol, propyl paraben and peppermint supreme flavor were added. Weighed amount of netupitant was added to the above mixture and dissolved to give a lipid phase.

Preparation of Aqueous Phase: 90% of required quantity of Milli-Q water per batch was taken in another manufacturing vessel in which weighed quantity of poloxamer-188 was added and dissolved by heating at 55° C. (50° C. to 60° C.). dextrose, sucralose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

Required quantity of palonosetron hydrochloride was added from prepared stock solution after high pressure homogenization. Finally, the pH of the preparation was adjusted in between 6.0-6.5 with tris base.

The size and zeta potential of the undiluted NLC dispersion were measured using particle size analyzer (Lite Sizer, Anton Paar).

The nano dispersion obtained was characterised for the description, pH, hydrodynamic diameter and the data is tabulated below:

TABLE 2
Characteristics data of the nanodispersion:

| Characteristics of the | | | |
|---|---|---|---|
| nanodispersion | 135A | 135C | 135D |
| Description | Off-white | Off-white | Off-white |
| | dispersion | dispersion | dispersion |
| pH | 6.49 | 6.62 | 6.62 |
| Osmolality (mOsm/kg) | 241 | 227 | 258 |
| Particle size distribution | | | |
| HDD (nm) | 132.24 | 111.18 | 114.28 |
| D10 (nm) | 69.61 | 57.54 | 64.45 |
| D50 (nm) | 128.80 | 115.88 | 116.75 |
| D90 (nm) | 223.65 | 197.43 | 186.70 |
| PDI (%) | 21.37 | 23.17 | 22.0 |
| SPAN | 1.19 | 1.21 | 1.05 |
| Zeta potential (mV) | −11.17 | −8.80 | −10.20 |
| Assay of netupitant (%) | 92.6 | 93.5 | 103.3 |

### Example 3: Oral Nanocarrier Formulation of Netupitant and Palonosetron with Various PEGylated Lipid Concentrations and Containing Cannabidiol

| | | Batch# | | |
|---|---|---|---|---|
| | | | 136D | 136E |
| | | 136A | (CBD: | (CBD: |
| | | (CBD: | 0.4%; | 0.4%; |
| | | 0.4%; | PEGylated | PEGylated |
| | | PEGylated | lipid: | lipid: |
| | | lipid: 0%) | 0.6%) | 1.2%) |
| S. No | Ingredients | %(w/v) | | |
| 1 | decanoic acid | 1.200 | 1.200 | 1.200 |
| 2 | Labrafac Lipophile WL | 1.200 | 1.200 | 1.200 |
| | 1349 (Medium Chain | | | |
| | Triglycerides NF) | | | |
| 3 | N-(Carbonyl- | 0.000 | 0.600 | 1.200 |
| | methoxypolyethylenglycol- | | | |
| | 2000)-1,2-distearoyl-sn- | | | |
| | glycero-3- | | | |
| | phosphoethanolamine, | | | |
| | MPEG-2000-DSPE, Na-salt | | | |
| | (Lipoid PE 18:0/18:0-PEG | | | |
| | 2000) | | | |
| 4 | cannabidiol (CBD) | 0.400 | 0.400 | 0.400 |
| 5 | netupitant | 0.600 | 0.600 | 0.600 |
| 6 | palonosetron hydrochloride | 0.001 | 0.001 | 0.001 |
| 7 | ethanol | 1.000 | 1.000 | 1.000 |
| 8 | Egg Lecithin (Lipoid E80S) | 1.000 | 1.000 | 1.000 |
| 9 | cholesterol | 0.300 | 0.300 | 0.300 |
| 10 | poloxamer-188 | 1.000 | 1.000 | 1.000 |
| 11 | vitamin E acetate | 0.020 | 0.020 | 0.020 |
| 12 | ascorbyl palmitate | 0.020 | 0.020 | 0.020 |
| 13 | propyl paraben | 0.020 | 0.020 | 0.020 |
| 14 | 1,4,7,10- | 0.002 | 0.002 | 0.002 |
| | Tetraazacyclododecane- | | | |
| | 1,4,7,10-tetraacetic acid | | | |
| | (DOTA) | | | |
| 15 | dextrose anhydrous | 3.500 | 3.500 | 3.500 |
| 16 | sucralose | 0.100 | 0.100 | 0.100 |
| 17 | peppermint supreme | 0.100 | 0.100 | 0.100 |
| 18 | tris base | 0.100 | 0.100 | 0.100 |
| 19 | Milli-Q Water | Q.S to | Q.S to | Q.S to |
| | | 100% | 100% | 100% |

### Manufacturing Procedure

Preparation of Oil Phase: Accurately weighed quantity of vitamin E acetate was taken into a manufacturing vessel. Labrafac Lipophile WL 1349 (oil) was subsequently added to it followed by decanoic acid and dissolved by heating on a water bath at 55° C. (50° C. to 60° C.). Weighed quantities of ascorbyl palmitate, cholesterol, PEG-PE and egg lecithin (Lipoid E80S) were further added and dissolved. This mixture was cooled to 40° C., ethanol, propyl paraben and peppermint supreme flavor were added. Weighed amount of netupitant and cannabidiol were added to the above mixture and dissolved to give a lipid phase.

Preparation of Aqueous Phase: 90% of required quantity of Milli-Q water per batch was taken in another manufacturing vessel in which weighed quantity of Poloxamer-188 was dissolved by heating at 55° C. (50° C. to 60° C.). Dextrose, sucralose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

Required quantity of palonosetron hydrochloride was added from prepared stock solution after high pressure homogenization. Finally, the pH of the preparation was adjusted in between 6.0-6.5 with tris base. The size and zeta potential of the undiluted NLC dispersion were measured using particle size analyzer (Lite Sizer, Anton Paar).

TABLE 3
Characteristics data of the nanodispersion
Characteristics of the nanodispersion

| Description | Off-white | Off-white | Off-white |
|---|---|---|---|
| | dispersion | dispersion | dispersion |
| pH | 6.57 | 6.68 | 6.70 |
| Osmolality (mOsm/kg) | 220 | 213 | 232 |
| Particle Size Distribution | | | |
| HDD (nm) | 214.01 | 135.62 | 158.03 |
| D10 (nm) | 134.18 | 67.07 | 79.96 |
| D50 (nm) | 196.82 | 136.58 | 166.52 |
| D90 (nm) | 284.31 | 244.0 | 277.88 |
| PDI (%) | 12.90 | 22.53 | 25.93 |
| SPAN | 0.76 | 1.29 | 1.19 |
| Zeta Potential (mV) | −28.50 | −13.87 | −9.73 |
| Assay of Netupitant (%) | 93.7 | 90.3 | 99.4 |
| Assay of CBD (%) | 90.1 | 93.8 | 97.0 |

As the concentration of PEG-PE increased in the NLC formulations, decrease in the Zeta Potential was noted which indicates effective shielding of NLC with PEG- PE. The Clarity of the NLC formulations increased with increase in the concentration of PEG-PE.

### Example 4: Preparation of cannabidiol-IH Formulation for Comparative Evaluation of Permeability

The example 1 of published patent #WO 2015/193668 A1 was reproduced in the laboratory. The composition reproduced from the patent for evaluating comparative permeability study is described below:

### Composition of CBD-IH Oral Preparation

| | | |
|---|---|---|
| Ingredients | CBD Patent* | CBD-IH |

| | | | |
|---|---|---|---|
| cannabidiol (CBD) | 100 | mg/mL | 100 mg/mL |
| anhydrous ethanol | 79.0 | mg/mL | 79.0 mg/mL |
| sucralose | 0.5 | mg/mL | 0.5 mg/mL |
| strawberry flavor | 0.2 | mg/mL | — |

| | | |
|---|---|---|
| sesame oil | q.s to 1.0 mL | sesame oil q.s to 1.0 mL |

The composition disclosed in WO 2015/193668 A1 (Example 1) was reproduced in the lab to be used as a control sample.

### Method of Preparation of CBD-IH

Weighed quantity of anhydrous ethanol was taken into a vial, to which cannabidiol and sucralose was added and dissolved. Required quantity of sesame oil was added to the alcoholic mixture and vortexed for 2 min to give a homogenous product.

### Permeation Study by In Vitro Non-Everted Rat Intestinal SAC Model

Permeation study was conducted by non-everted rat intestinal sac model. The noneverted gut sac method involves placing the test sample in the rat gut sac without everting. The non-everted gut sac was prepared by removing the small intestine section from overnight fasted anaesthetised rats. The intestine was excised and rinsed with ringer lactate solution at room temperature under aeration. The jejunum was knotted at one end, filled with aliquot volume of test sample and the other end was knotted. This intestine bag or sac (considered as donor compartment) was placed in a buffer solution (receptor compartment) maintained at 37° C. and at 100 rpm.
(1) Volume of test sample taken into the Intestinal sac i.e., donor compartment—2 mL
(2) Volume of the buffer medium taken in receptor compartment—75 mL
(3) Buffer solution (receptor compartment)—pH 6.8 Phosphate buffer (With 1% (w/v) Solutol HS15)
(4) Time Points at which samples were collected—0.25 h, 0.5h, 1 h, 2 h, 3 h, 4 h, 6 h, 9 h, 12 h and 24 h.
(5) Volume of the sample collected at each time point—10 mL

TABLE 4
Test samples used in the permeation study

| S. No | Batch # | Description |
|---|---|---|
| 1 | B#136G | Akynzeo* (Used as control) |
| 2 | B#119C | CBD Oral Preparation (CBD-IH)** (Used as control) |
| 3 | B#135A | NEPA NLC with 0% PEGylated lipid |
| 4 | B#135C | NEPA NLC with 0.6% PEGylated lipid |
| 5 | B#135D | NEPA NLC with 1.2% PEGylated lipid |
| 6 | B#136A | NEPA + cannabidiol NLC with 0% PEGylated lipid |
| 7 | B#136D | NEPA + cannabidiol NLC with 0.6% PEGylated lipid |
| 8 | B#136E | NEPA + cannabidiol NLC with 1.2% PEGylated lipid |
| 9 | B#136C | NEPA self-dispersible NLC concentrate*** with 4.6% |
| | | PEGylated lipid |

Note:
*Akynzeo capsule (Batch No: 39002435; Expiry Date: February 2022) was completely dispersed in 50 mL volumetric flask with pH 6.8 Phosphate buffer by vortex mixing.
**CBD Oral Preparation (CBD-IH) (40 μL was taken and was dispersed in pH 6.8 Phosphate buffer, volume made up to 2 mL).
***(130 mg of SDNLC (self-dispersible nano lipid carrier system) was taken and dispersed in pH 6.8 Phosphate buffer, volume made up to 2 mL).

The samples were withdrawn from the acceptor compartment at the predetermined time intervals. The samples were analyzed by HPLC and cumulative amount of active ingredient released from unit surface area of intestine was calculated. A graph was plotted between cumulative amount of active ingredient released from unit surface area of intestine and time. The slope of the graph gives the permeation flux rate in (mcg/cm²/h).

The flux rate was calculated for netupitant from NEPA NLC and for both netupitant and CBD from NEPA+CBD NLC which are tabulated in Table 5 to Table 8.

Method for Determination of Entrapment Efficiency: 2 mL of NLC preparation was taken into 100 KD MWCO Filter and centrifuged at 4500 rpm for 1h. The upper lipid layer obtained was suitably diluted in assay diluent and the amount of active ingredient entrapped in the lipid was determined by HPLC.

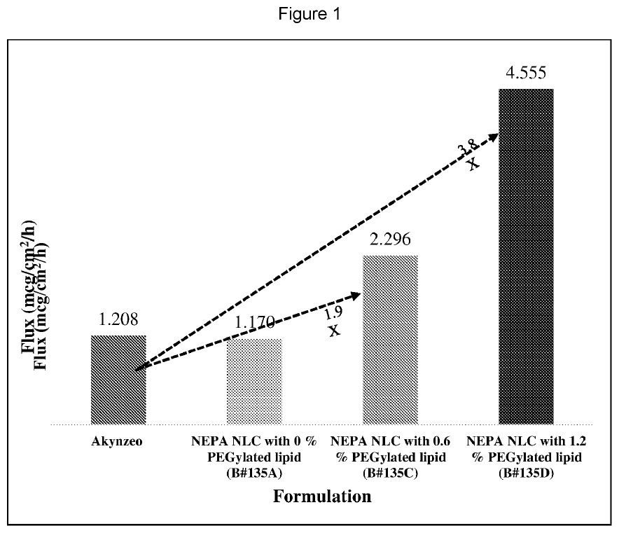

FIG. 1 shows that the permability of netupitant from NLC carrier devoid of PEGylated lipid is comparable to Akynzeo. Surprisingly, the permeability of netupitant in presence of PEGylated lipid is increased by 2 to 4 times when compared to Akynzeo and NLC preparation devoid of PEGylated lipid.

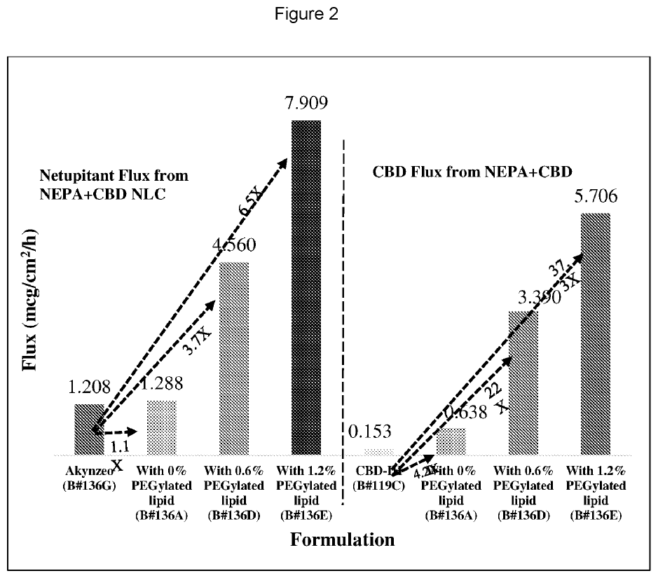

FIG. 2 shows that the permeability of CBD with NLC carrier devoid of PEGylated lipid is improved by about 4 times when compared to the prior art formulation. Surprisingly, a marked increase in permeation rate of CBD was observed when PEGylated lipid is used in the preparation. In presence of PEGylated lipid in NLC carrier system, the permeability of CBD increased by about 22-37 times than prior art formulation and about 5-9 times than NLC preparation devoid of PEGylated Lipid. This clearly demonstrates that PEGylated lipid in NLC carrier increased permeability of active ingredients.

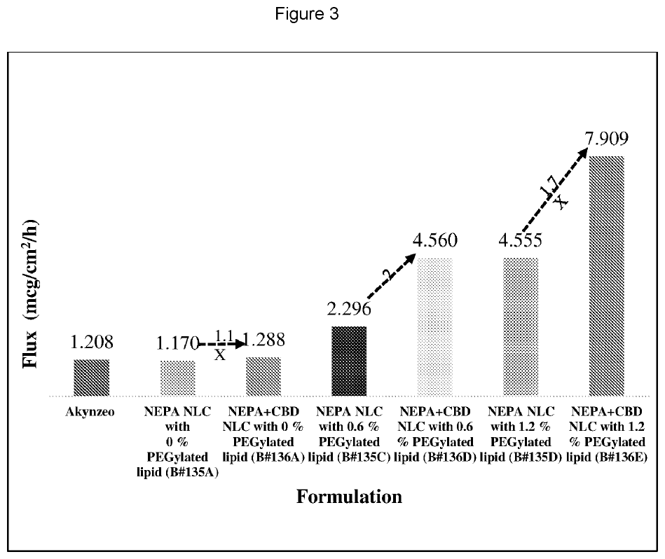

FIG. 3 shows that surprisingly, it is also found that in presence of CBD and PEGylated lipid the permeability of netupitant is increased about 2 fold than compared to corresponding formulations that contained PEGylated lipid and devoid of CBD. The permeability of netupitant from NLC formulation comprising of CBD and PEGylated lipid was about 4 fold more than NLC formulation that did not contain CBD & PEGylated lipid. This clearly demonstrates that (a) PEGylated lipid in NLC formulation increased permeability of Cannabidiol (CBD) significantly, (b) PEGylated lipid and CBD in NLC formulation synergistically enhance permeability of active ingredients.

TABLE 5
Comparative permeability flux of netupitant from
formulations

| | | | R² | | | |
|---|---|---|---|---|---|---|
| | | | value | | | |
| | | netupitant | from | | | |
| | | flux rate | the | EE | HDD | ZP |
| S. No | Formulation | (mcg/cm²/h) | graph | (%) | (nm) | (mV) |
| 1 | Akynzeo | 1.208 | 0.99 | NA | 1396.17 | −10.13 |
| | (B#136G) | | | | | |
| 2 | NEPA NLC | 1.170 | 0.85 | 93.4 | 132.24 | −11.17 |
| | with 0 % | | | | | |
| | PEGylated lipid | | | | | |
| | (B#135A) | | | | | |
| 3 | NEPA NLC | 2.296 | 0.97 | 88.8 | 111.18 | −8.80 |
| | with 0.6 % | | | | | |
| | PEGylated lipid | | | | | |
| | (B#135C) | | | | | |
| 4 | NEPA NLC | 4.555 | 0.93 | 84.3 | 114.28 | −10.20 |
| | with 1.2 % | | | | | |
| | PEGylated lipid | | | | | |
| | (B#135D) | | | | | |

EE: entrapment efficiency;
Zp: zetapotential;
HDD: hydrodynamic diameter

TABLE 6
Comparative permeability flux of netupitant and CBD from
formulations

| | | | | cannabidiol | | | | | |
|---|---|---|---|---|---|---|---|---|---|
| | | netupitant | | (CBD) | | | | | |
| | | Flux | R2 | Flux | R2 | | | | |
| | | rate | value | rate | value | | | | |
| | | (mcg/ | from | (mcg/ | from | | | | |
| | | cm2/ | the | cm2/ | the | EE (%) | | HDD | Zp |
| S. No | Formulation | h) | graph | h) | graph | netupitant | cannabidiol | (nm) | (mV) |
| 1 | Akynzeo | 1.2 | 0.98 | NA | NA | NA | NA | 1396.1 | −10.1 |
| | (B#136G) | | | | | | | | |
| 2 | CBD | NA | NA | 0.15 | 0.86 | NA | NA | NA | NA |
| | Oral | | | | | | | | |
| | Preparation | | | | | | | | |
| | (CBD-IH) | | | | | | | | |
| | (B#119C) | | | | | | | | |
| 3 | NEPA + CBD | 1.2 | 0.97 | 0.63 | 0.96 | 94.1 | 87.4 | 214.0 | −28.5 |
| | NLC with | | | | | | | | |
| | 0% | | | | | | | | |
| | PEGylated | | | | | | | | |
| | lipid | | | | | | | | |
| | (B#136A) | | | | | | | | |
| 4 | NEPA + CBD | 4.5 | 0.85 | 3.39 | 0.87 | 89.5 | 85.6 | 135.6 | −13.8 |
| | NLC with | | | | | | | | |
| | 0.6% | | | | | | | | |
| | PEGylate | | | | | | | | |
| | dlipid | | | | | | | | |
| | (B#136D) | | | | | | | | |
| 5 | NEPA + CBD | 7.9 | 0.98 | 5.70 | 0.99 | 98.2 | 93.1 | 158.0 | −9.73 |
| | NLC with | | | | | | | | |
| | 1.2% | | | | | | | | |
| | PEGylate | | | | | | | | |
| | dlipid | | | | | | | | |
| | (B#136E) | | | | | | | | |

EE: entrapment efficiency;
Zp: zetapotential;
HDD: hydrodynamic diameter

TABLE 7
Summary of comparative permeability of netupitant from
different NLC formulations

| | | Netupitant | | | | |
|---|---|---|---|---|---|---|
| | | | R2 | | | |
| | | | value | | | |
| | | | from | | | |
| | | Flux rate | the | Netupitant | HDD | Zp |
| S. No | Formulation | (mcg/cm²/h) | graph | EE (%) | (nm) | (mV) |
| 1 | Akynzeo | 1.208 | 0.999 | NA | 1396.17 | −10.13 |
| | (B#136G) | | | | | |
| 2 | NEPA NLC | 1.170 | 0.855 | 93.4 | 132.24 | −11.17 |
| | with 0% | | | | | |
| | PEGylated | | | | | |
| | lipid | | | | | |
| | (B#135A) | | | | | |
| 3 | NEPA + CBD | 1.288 | 0.979 | 94.1 | 214.01 | −28.50 |
| | NLC with | | | | | |
| | 0% | | | | | |
| | PEGylated | | | | | |
| | lipid | | | | | |
| | (B#136A) | | | | | |
| 4 | NEPA NLC | 2.296 | 0.979 | 88.8 | 111.18 | −8.80 |
| | with 0.6% | | | | | |
| | PEGylated | | | | | |
| | lipid | | | | | |
| | (B#135C) | | | | | |
| 5 | NEPA + CBD | 4.560 | 0.850 | 89.5 | 135.62 | −13.87 |
| | NLC with | | | | | |
| | 0.6% | | | | | |
| | PEGylated | | | | | |
| | lipid | | | | | |
| | (B#136D) | | | | | |
| 6 | NEPA NLC | 4.555 | 0.930 | 84.3 | 114.28 | −10.20 |
| | with 1.2% | | | | | |
| | PEGylated | | | | | |
| | lipid | | | | | |
| | (B#135D) | | | | | |
| 7 | NEPA + CBD | 7.909 | 0.982 | 98.2 | 158.03 | −9.73 |
| | NLC with | | | | | |
| | 1.2% | | | | | |
| | PEGylated | | | | | |
| | lipid | | | | | |
| | (B#136E) | | | | | |

EE: entrapment efficiency;
Zp: zetapotential;
HDD: hydrodynamic diameter

TABLE 8
Data representing the synergistic effect of PEG-PE and CBD
on the permeation of netupitant

| | | | | NEPA | CBD | | |
|---|---|---|---|---|---|---|---|
| | | | | flux | flux | | |
| | | PEGylated | | rate | rate | | CBD |
| | Formulation | lipid | CBD | (mcg/ | (mcg/ | netupitant | flux |
| S. No | details | (% w/v) | (% w/v) | cm²/h) | cm²/h) | flux change | change |
| 1 | Akynzeo | Nil | Nil | 1.208 | Nil | — | — |
| | (control) | | | | | | |
| 2 | B#119C | Nil | 10% | Nil | 0.153 | — | — |
| | CBD-IH | | | | | | |
| | (control) | | | | | | |
| 3 | B#135A | 0% | Nil | 1.170 | Nil | Comparable | — |
| | NEPA | | | | | to Akynzeo | |
| | NLC | | | | | | |
| | without | | | | | | |
| | PEGylated | | | | | | |
| | lipid | | | | | | |
| 4 | B#135C | 0.6% | Nil | 2.296 | Nil | 1.9 times > | — |
| | NEPA | | | | | than | |
| | NLC | | | | | Akynzeo | |
| | with | | | | | | |
| | PEGylated | | | | | | |
| | lipid | | | | | | |
| 5 | B#135D | 1.2% | Nil | 4.555 | Nil | 3.77 times > | — |
| | NEPA | | | | | than | |
| | NLC | | | | | Akynzeo | |
| | with | | | | | | |
| | PEGylated | | | | | | |
| | lipid | | | | | | |
| 6 | B#136A | 0% | 0.4% | 1.288 | 0.638 | Comparable | 4.1 |
| | NEPA | | | | | to | times > |
| | NLC | | | | | Akynzeo | than |
| | without | | | | | | CBD-IH |
| | PEGylated | | | | | | |
| | lipid | | | | | | |
| 7 | B#136D | 0.6% | 0.4% | 4.560 | 3.390 | 3.7 times > | 22.1 |
| | NEPA | | | | | than | times > |
| | NLC | | | | | Akynzeo | than |
| | with | | | | | | CBD-IH > |
| | PEGylated | | | | | | than |
| | lipid | | | | | | |
| 8 | B#136E | 1.2% | 0.4% | 7.909 | 5.706 | 6.5 times > | 37.3 |
| | NEPA | | | | | than | times > |
| | NLC | | | | | Akynzeo | than |
| | with | | | | | | CBD-IH |
| | PEGylated | | | | | | |
| | Lipid | | | | | | |

### B. Self-Dispersible Nanocarrier Concentrate Compositions in Capsules

### Example 5: CBD Oral Self-Dispersible NLC Concentrate

| | | Quantity | |
|---|---|---|---|
| S. No | Ingredients | (mg) | % w/v |
| 1 | cannabidiol synthetic | 150 | 28.04 |
| 2 | decanoic acid | 75 | 14.02 |
| 3 | egg lecithin | 50 | 9.35 |
| 4 | N-(Carbonyl-methoxypolyethylenglycol- | 30 | 5.61 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 5 | Labrafac Lipophile WL 1349 | 100 | 18.69 |
| | (Medium Chain Triglycerides NF) | | |
| 6 | Kolliphor RH40 | 95 | 17.76 |

### Manufacturing Procedure

Accurately weighed quantity of Labrafac Lipophile WL 1349 (Medium Chain Triglycerides NF) and Kolliphor RH 40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous matrix.

The obtained mixture can be encapsulated in hard or soft gelatin capsules.

### Example 6: CBD Oral Self-Dispersible NLC Concentrate

| | | Quantity | |
|---|---|---|---|
| S. No | Ingredients | (mg) | % w/v |
| 1 | cannabidiol synthetic | 150 | 28.04 |
| 2 | decanoic acid | 75 | 14.02 |
| 3 | egg lecithin | 50 | 9.35 |
| 4 | N-(Carbonyl-methoxypolyethylenglycol- | 30 | 5.61 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 5 | caprylic acid | 100 | 18.69 |
| 6 | Kolliphor RH40 | 95 | 17.76 |

### Manufacturing Procedure

Accurately weighed quantity of caprylic acid and Kolliphor RH 40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous matrix.

The obtained mixture can be encapsulated in hard or soft gelatin capsules.

### Example 7: CBD Oral Self-Dispersible NLC concentrate

| | | Quantity | |
|---|---|---|---|
| S. No | Ingredients | (mg) | % w/v |
| 1 | cannabidiol synthetic | 150 | 28.04 |
| 2 | decanoic acid | 75 | 14.02 |
| 3 | egg lecithin | 50 | 9.35 |
| 4 | N-(Carbonyl-methoxypolyethylenglycol- | 30 | 5.61 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 5 | Incromega E3322 (Fish Oil) | 100 | 18.69 |
| 6 | Kolliphor RH40 | 95 | 17.76 |

### Manufacturing Procedure

Accurately weighed quantity of Incromega E3322 and Kolliphor RH 40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous matrix.

The obtained mixture can be encapsulated in hard or soft gelatin capsules.

### Example 8: Example of CBD Oral Self-Dispersible NLC Concentrate

| | | Quantity | |
|---|---|---|---|
| S. No | Ingredients | (mg) | % w/v |
| 1 | cannabidiol synthetic | 150 | 28.04 |
| 2 | decanoic acid | 75 | 14.02 |
| 3 | egg lecithin | 50 | 9.35 |
| 4 | N-(Carbonyl-methoxypolyethylenglycol- | 30 | 5.61 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 5 | eicosapentaenoic acid (EPA) | 100 | 18.69 |
| 6 | Kolliphor RH40 | 95 | 17.76 |

### Manufacturing Procedure

Accurately weighed quantity of eicosapentaenoic acid (EPA) and Kolliphor RH 40 were taken into a glass vial to which decanoic acid, PEGPE and egg lecithin were added and the contents were dissolved by heating on a water bath at 40° C. Then, required quantity of cannabidiol was added to the above mixture and dissolved by further heating at 40° C. to give a homogenous matrix.

The obtained mixture can be encapsulated in hard or soft gelatin capsules.

### Example 9: CBD Oral Self-Dispersible NLC Concentrate

| | | Quantity | |
|---|---|---|---|
| S. No | Ingredients | (mg) | (% w/v) |
| 1 | cannabidiol synthetic | 150 | 28.04 |
| 2 | decanoic acid | 75 | 14.02 |
| 3 | egg lecithin | 50 | 9.35 |
| 4 | N-(Carbonyl-methoxypolyethylenglycol- | 30 | 5.61 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 5 | docosahexaenoic acid (DHA) | 100 | 18.69 |
| 6 | Kolliphor RH40 | 95 | 17.76 |

### Manufacturing Procedure

Accurately weighed quantity of docosahexaenoic acid (DHA) and Kolliphor RH 40 were taken into a glass vial to which decanoic acid, PEGPE and egg lecithin were added and the contents were dissolved by heating on a water bath at 40° C. Then, required quantity of cannabidiol was added to the above mixture and dissolved by further heating at 40° C. to give a homogenous matrix.

The obtained mixture can be encapsulated in hard or soft gelatin capsules.

### C. Orally Administered Dispersible Liquid Oil Compositions

### Example 10: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 0.3 | 0.03 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 155 | 14.49 |
| | Chain Triglycerides NF) | | |

### Manufacturing Procedure

Accurately weighed quantity of Labrafac Lipophile WL 1349 (Medium Chain Triglycerides NF), grapeseed oil and Kolliphor RH40 were taken into a glass vial to which decanoic acid, PEG-PE and Egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 11: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 125 | 11.68 |
| | Chain Triglycerides NF) | | |

### Manufacturing Procedure

Accurately weighed quantity of Labrafac Lipophile WL 1349 (Medium Chain Triglycerides NF), grapeseed oil and Kolliphor RH40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 12: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 100 | 9.35 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 55 | 5.14 |
| | Chain Triglycerides NF) | | |

### Manufacturing Procedure

Accurately weighed quantity of Labrafac Lipophile WL 1349 (Medium Chain Triglycerides NF), grapeseed oil and Kolliphor RH40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 13: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 0.3 | 0.03 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | sucrose laurate | 100 | 9.35 |
| 13 | Labrafac Lipophile WL 1349 (Medium | 155 | 14.49 |
| | Chain Triglycerides NF) | | |

### Manufacturing Procedure

Accurately weighed quantity of Labrafac Lipophile WL 1349 (Medium Chain Triglycerides NF) and grapeseed oil were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which sucrose laurate, neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 14: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | sucrose laurate | 100 | 9.35 |
| 13 | Labrafac Lipophile WL 1349 | 125 | 11.68 |
| | (Medium Chain Triglycerides NF) | | |

### Manufacturing Procedure

Accurately weighed quantity of Labrafac Lipophile WL 1349 (Medium Chain Triglycerides NF) and grapeseed oil were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which sucrose laurate, neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 15: Self-Dispersible Cannabidiol Oral Liquid Oil

| | | Quantity | |
|---|---|---|---|
| S. No | Ingredients | (mg) | % w/v |
| 1 | cannabidiol synthetic | 150 | 14.02 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 100 | 9.35 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | sucrose laurate | 100 | 9.35 |
| 13 | Labrafac Lipophile WL 1349 (Medium | 55 | 5.14 |
| | Chain Triglycerides NF) | | |

### Manufacturing Procedure

Accurately weighed quantity of Labrafac Lipophile WL 1349 (Medium Chain Triglycerides NF) and grapeseed oil were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which sucrose laurate, neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 16: Self-Dispersible Netupitant and Palonosetron Oral Liquid Oil

| | | Quantity | |
|---|---|---|---|
| S. No | Ingredients | (mg) | % w/v |
| 1 | netupitant | 100 | 9.35 |
| 2 | palonosetron hydrochloride | 0.167 | 0.02 |
| 3 | grapeseed oil | 300 | 28.04 |
| 4 | decanoic acid | 50 | 4.67 |
| 5 | egg lecithin | 50 | 4.67 |
| 6 | N-(Carbonyl-ethoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | propylene glycol | 25 | 2.34 |
| 14 | Tween-80 | 100 | 9.35 |
| 15 | Labrafac Lipophile WL 1349 (Medium | 150 | 14.02 |
| | Chain Triglycerides NF) | | |

### Manufacturing Procedure

Accurately weighed quantity of Labrafac Lipophile WL 1349 (Medium Chain Triglycerides NF), Tween-80 and grapeseed oil were taken into a glass vial to which decanoic acid, PEG-PE and egg lecithin were added and the contents were dissolved by heating on a water bath at 40° C. Then, required quantity of netupitant was added to the above mixture and dissolved by further heating at 40° C. to give a homogenous oil matrix which was then cooled to room temperature.

In another vial weighed quantity of propylene glycol was taken and palonosetron hydrochloride was dissolved in it followed by ethanol to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 17: Self-Dispersible Netupitant, Cannabidiol and Palonosetron Oral Liquid Oil

| | | Quantity | |
|---|---|---|---|
| S. No | Ingredients | (mg) | % w/v |
| 1 | netupitant | 100 | 9.35 |
| 2 | palonosetron hydrochloride | 0.167 | 0.02 |
| 3 | cannabidiol | 50 | 4.67 |
| 4 | grapeseed oil | 300 | 28.04 |
| 5 | decanoic acid | 50 | 4.67 |
| 6 | egg lecithin | 50 | 4.67 |
| 7 | N-(Carbonyl-methoxypolyethylenglycol- | 0.3 | 0.03 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 8 | neotame | 30 | 2.80 |
| 9 | peppermint supreme | 20 | 1.87 |
| 10 | spearmint oil | 20 | 1.87 |
| 11 | cinnamon oil | 15 | 1.40 |
| 12 | menthol | 10 | 0.93 |
| 13 | ethanol | 100 | 9.35 |
| 14 | propylene glycol | 25 | 2.34 |
| 15 | Tween-80 | 100 | 9.35 |
| 16 | Labrafac Lipophile WL 1349 (Medium | 130 | 12.15 |
| | Chain Triglycerides NF) | | |

### Manufacturing Procedure

Accurately weighed quantity of Labrafac Lipophile WL 1349 (Medium Chain Triglycerides NF), Tween-80 and grapeseed oil were taken into a glass vial to which decanoic acid, PEG-PE and egg lecithin were added and the contents were dissolved by heating on a water bath at 40° C. Then, required quantity of cannabidiol followed by netupitant were added to the above mixture and dissolved by further heating at 40° C. to give a homogenous oil matrix which was then cooled to room temperature.

In another vial weighed quantity of propylene glycol was taken and palonosetron hydrochloride was dissolved in it followed by ethanol to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 18: Self-Dispersible Netupitant, Cannabidiol and Palonosetron Oral Liquid Oil

| | | Quantity | |
|---|---|---|---|
| S. No | Ingredients | (mg) | % w/v |
| 1 | netupitant | 100 | 9.35 |
| 2 | palonosetron hydrochloride | 0.167 | 0.02 |
| 3 | cannabidiol | 50 | 4.67 |
| 4 | grapeseed oil | 300 | 28.04 |
| 5 | decanoic acid | 50 | 4.67 |
| 6 | egg lecithin | 50 | 4.67 |
| 7 | N-(Carbonyl-methoxypolyethylenglycol- | 30 | 2.80 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 8 | neotame | 30 | 2.80 |
| 9 | peppermint supreme | 20 | 1.87 |
| 10 | spearmint oil | 20 | 1.87 |
| 11 | cinnamon oil | 15 | 1.40 |
| 12 | menthol | 10 | 0.93 |
| 13 | ethanol | 100 | 9.35 |
| 14 | propylene glycol | 25 | 2.34 |
| 15 | Tween-80 | 100 | 9.35 |
| 16 | Labrafac Lipophile WL 1349 (Medium | 100 | 9.35 |
| | Chain Triglycerides NF) | | |

### Manufacturing Procedure

Accurately weighed quantity of Labrafac Lipophile WL 1349 (Medium Chain Triglycerides NF), Tween-80 and grapeseed oil were taken into a glass vial to which decanoic acid, PEG-PE and egg lecithin were added and the contents were dissolved by heating on a water bath at 40° C. Then, required quantity of cannabidiol followed by netupitant were added to the above mixture and dissolved by further heating at 40° C. to give a homogenous oil matrix which was then cooled to room temperature.

In another vial weighed quantity of propylene glycol was taken and palonosetron hydrochloride was dissolved in it followed by ethanol to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 19: Self-Dispersible Netupitant, Cannabidiol and Palonosetron Oral Liquid Oil

| | | Quantity | |
|---|---|---|---|
| S. No | Ingredients | (mg) | % w/v |
| 1 | netupitant | 100 | 9.35 |
| 2 | palonosetron hydrochloride | 0.167 | 0.02 |
| 3 | cannabidiol | 50 | 4.67 |
| 4 | grapeseed oil | 300 | 28.04 |
| 5 | decanoic acid | 50 | 4.67 |
| 6 | egg lecithin | 50 | 4.67 |
| 7 | N-(Carbonyl-methoxypolyethylenglycol- | 100 | 9.35 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 8 | neotame | 30 | 2.80 |
| 9 | peppermint supreme | 20 | 1.87 |
| 10 | spearmint oil | 20 | 1.87 |
| 11 | cinnamon oil | 15 | 1.40 |
| 12 | menthol | 10 | 0.93 |
| 13 | ethanol | 100 | 9.35 |
| 14 | propylene glycol | 25 | 2.34 |
| 15 | Tween-80 | 100 | 9.35 |
| 16 | Labrafac Lipophile WL 1349 (Medium | 30 | 2.80 |
| | Chain Triglycerides NF) | | |

### Manufacturing Procedure

Accurately weighed quantity of Labrafac Lipophile WL 1349 (Medium Chain Triglycerides NF), Tween-80 and grapeseed oil were taken into a glass vial to which decanoic acid, PEG-PE and egg lecithin were added and the contents were dissolved by heating on a water bath at 40° C. Then, required quantity of cannabidiol followed by netupitant were added to the above mixture and dissolved by further heating at 40° C. to give a homogenous oil matrix which was then cooled to room temperature.

In another vial weighed quantity of propylene glycol was taken and palonosetron hydrochloride was dissolved in it followed by ethanol to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 20: Self Dispersible Gabapentin Oral Liquid Oil

| | | Quantity | |
|---|---|---|---|
| S. No | Ingredients | (mg) | % w/v |
| 1 | gabapentin | 100 | 9.35 |
| 2 | omega fatty acid | 475 | 44.39 |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Tween-80 | 100 | 9.35 |

### Manufacturing Procedure

Accurately weighed quantity of omega fatty acid and Tween-80 were taken into a glass vial to which decanoic acid, PEG-PE and egg lecithin were added and the contents were dissolved by heating on a water bath at 40° C. Then, required quantity of gabapentin was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 21: Self Dispersible Gabapentin Oral Liquid Oil

| | | Quantity | |
|---|---|---|---|
| S. No | Ingredients | (mg) | % w/v |
| 1 | gabapentin | 100 | 9.35 |
| 2 | cannabidiol | 50 | 4.67 |
| 3 | omega fatty acid | 425 | 39.72 |
| 4 | decanoic acid | 50 | 4.67 |
| 5 | egg lecithin | 50 | 4.67 |
| 6 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Tween-80 | 100 | 9.35 |

### Manufacturing Procedure

Accurately weighed quantity of omega fatty acid and Tween-80 were taken into a glass vial to which decanoic acid, PEG-PE and egg lecithin were added and the contents were dissolved by heating on a water bath at 40° C. Then, required quantity of cannabidiol followed by gabapentin were added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 22: Self-Dispersible Atorvastatin Oral Formulation

| | | Quantity | |
|---|---|---|---|
| S. No | Ingredients | (mg) | % w/v |
| 1 | atorvastatin | 80 | 7.48 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 195 | 18.22 |
| | Chain Triglycerides NF) | | |

### Manufacturing Procedure

Accurately weighed quantity of Labrafac Lipophile WL 1349 (Medium Chain Triglycerides NF) Kolliphor RH40 and grapeseed oil were taken into a glass vial to which decanoic acid, PEG-PE and egg lecithin were added and the contents were dissolved by heating on a water bath at 40° C. Then, required quantity of atorvastatin was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 23: Self-Dispersible Atorvastatin Oral Formulation

| | | Quantity | |
|---|---|---|---|
| S. No | Ingredients | (mg) | % w/v |
| 1 | atorvastatin | 80 | 7.48 |
| 2 | cannabidiol | 100 | 9.35 |
| 3 | grapeseed oil | 300 | 28.04 |
| 4 | decanoic acid | 50 | 4.67 |
| 5 | egg lecithin | 50 | 4.67 |
| 6 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Kolliphor RH40 | 100 | 9.35 |
| 14 | Labrafac Lipophile WL 1349 (Medium | 95 | 8.88 |
| | Chain Triglycerides NF) | | |

### Manufacturing Procedure

Accurately weighed quantity of Labrafac Lipophile WL 1349 (Medium Chain Triglycerides NF) Kolliphor RH40 and grapeseed oil were taken into a glass vial to which decanoic acid, PEG-PE, egg lecithin and cannabidiol were added and the contents were dissolved by heating on a water bath at 40° C. Then, required quantity of atorvastatin was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 24: Self-Dispersible Duloxetine Oral Liquid Oil

| | | Quantity | |
|---|---|---|---|
| S. No | Ingredients | (mg) | % w/v |
| 1 | duloxetine | 60 | 5.61 |
| 2 | Labrafac Lipophile WL 1349 (Medium | 515 | 48.13 |
| | Chain Triglycerides NF) | | |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Tween-80 | 100 | 9.35 |

### Manufacturing Procedure

Accurately weighed quantity of Labrafac Lipophile WL 1349 (Medium Chain Triglycerides NF)and Tween-80 were taken into a glass vial to which decanoic acid, PEG-PE and egg lecithin were added and the contents were dissolved by heating on a water bath at 40° C. Then, required quantity of duloxetine was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The oils used in the above formulations can be corn oil, coconut oil, fish oil, EPA, DHA, soybean oil, olive oil, flax seed oil, avocado oil, ginger oil, hemp seed oil, and grapeseed oil.

### Example 25: Self-Dispersible Duloxetine Oral Liquid Oil

| | | Quantity | |
|---|---|---|---|
| S. No | Ingredients | (mg) | % w/v |
| 1 | duloxetine | 60 | 5.61 |
| 2 | cannabidiol | 50 | 4.67 |
| 3 | Labrafac Lipophile WL 1349 (Medium | 465 | 43.46 |
| | Chain Triglycerides NF) | | |
| 4 | decanoic acid | 50 | 4.67 |
| 5 | egg lecithin | 50 | 4.67 |
| 6 | N-(Carbonyl- | 30 | 2.80 |
| | methoxypolyethylenglycol-2000)-1,2- | | |
| | distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Tween-80 | 100 | 9.35 |

### Manufacturing Procedure

Accurately weighed quantity of Labrafac Lipophile WL 1349 (Medium Chain Triglycerides NF)and Tween-80 were taken into a glass vial to which decanoic acid, PEG-PE and egg lecithin were added and the contents were dissolved by heating on a water bath at 40° C. Then, required quantity of cannabidiol followed by duloxetine was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The oils used in the above formulations can be corn oil, coconut oil, fish oil, EPA, DHA, soybean oil, olive oil, flax seed oil, avocado oil, ginger oil, hemp seed oil, and grapeseed oil.

### Example 26: Self-Dispersible Naproxen Sodium Oral Formulation

| | | Quantity | |
|---|---|---|---|
| S. No | Ingredients | (mg) | % w/v |
| 1 | naproxen sodium | 100 | 9.35 |
| 2 | grapeseed oil | 300 | 28.04 |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000-DSPE, | | |
| | Na-salt (Lipoid PE 18:0/18:0-PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 175 | 16.36 |
| | Chain Triglycerides NF) | | |

### Manufacturing Procedure

Accurately weighed quantity of Labrafac Lipophile WL 1349 (Medium Chain Triglycerides NF) Kolliphor RH40 and grapeseed oil were taken into a glass vial to which decanoic acid, PEG-PE and egg lecithin were added and the contents were dissolved by heating on a water bath at 40° C. Then, required quantity of naproxen sodium was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 27: Self-Dispersible Naproxen Sodium Oral Formulation

| | | Quantity | |
|---|---|---|---|
| S. No | Ingredients | (mg) | % w/v |
| 1 | naproxen sodium | 100 | 9.35 |
| 2 | cannabidiol | 50 | 4.67 |
| 3 | grapeseed oil | 300 | 28.04 |
| 4 | decanoic acid | 50 | 4.67 |
| 5 | egg lecithin | 50 | 4.67 |
| 6 | N-(Carbonyl- | 30 | 2.80 |
| | methoxypolyethylenglycol-2000)-1,2- | | |
| | distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Kolliphor RH40 | 100 | 9.35 |
| 14 | Labrafac Lipophile WL 1349 (Medium | 125 | 11.68 |
| | Chain Triglycerides NF) | | |

### Manufacturing Procedure

Accurately weighed quantity of Labrafac Lipophile WL 1349 (Medium Chain Triglycerides NF) Kolliphor RH40 and grapeseed oil were taken into a glass vial to which decanoic acid, PEG-PE, egg lecithin and cannabidiol were added and the contents were dissolved by heating on a water bath at 40° C. Then, required quantity of naproxen sodium was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 28: Self-Dispersible Abiraterone Acetate Oral Formulation

| | | Quantity | |
|---|---|---|---|
| S. No | Ingredients | (mg) | % w/v |
| 1 | abiraterone acetate | 100 | 9.35 |
| 2 | Labrafac Lipophile WL 1349 (Medium | 475 | 44.39 |
| | Chain Triglycerides NF) | | |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |

### Manufacturing Procedure

Accurately weighed quantity of Labrafac Lipophile WL 1349 (Medium Chain Triglycerides NF) and Kolliphor RH40 were taken into a glass vial to which decanoic acid, PEG-PE and egg lecithin were added and the contents were dissolved by heating on a water bath at 40° C. Then, required quantity of abiraterone acetate was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The concentration of Lipoid PE 18:0/18:0-PEG 2000 in the formulation may vary from 0 to 6% of the total formulation in the case of self-dispersible nano-carrier concentrate to be filled in capsules.

### Example 29: Self-Dispersible Abiraterone Acetate Oral Formulation

| | | Quantity | |
|---|---|---|---|
| S. No | Ingredients | (mg) | % w/v |
| 1 | abiraterone acetate | 100 | 9.35 |
| 2 | cannabidiol | 50 | 4.67 |
| 3 | Labrafac Lipophile WL 1349 (Medium | 425 | 39.72 |
| | Chain Triglycerides NF) | | |
| 4 | decanoic acid | 50 | 4.67 |
| 5 | egg lecithin | 50 | 4.67 |
| 6 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Kolliphor RH40 | 100 | 9.35 |

### Manufacturing Procedure

Accurately weighed quantity of Labrafac Lipophile WL 1349 (Medium Chain Triglycerides NF) and Kolliphor RH40 were taken into a glass vial to which decanoic acid, PEG-PE, egg lecithin and cannabidiol were added and the contents were dissolved by heating on a water bath at 40° C. Then, required quantity of abiraterone acetate was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The concentration of Lipoid PE 18:0/18:0-PEG 2000 in the formulation may vary from 0 to 6% of the total formulation in the case of self-dispersible nanocarrier concentrate to be filled in capsules.

### Example 30: Self-Dispersible Cabazitaxel Oral Formulation

| | | Quantity | |
|---|---|---|---|
| S. No | Ingredients | (mg) | % w/v |
| 1 | cabazitaxel | 120 | 11.21 |
| 2 | Labrafac Lipophile WL 1349 (Medium | 455 | 42.52 |
| | Chain Triglycerides NF) | | |
| 3 | decanoic acid | 50 | 4.67 |
| 4 | egg lecithin | 50 | 4.67 |
| 5 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |

### Manufacturing Procedure

Accurately weighed quantity of Labrafac Lipophile WL 1349 (Medium Chain Triglycerides NF) and Kolliphor RH40 were taken into a glass vial to which decanoic acid, PEG-PE and egg lecithin were added and the contents were dissolved by heating on a water bath at 40° C. Then, required quantity of cabazitaxel was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The concentration of Lipoid PE 18:0/18:0-PEG 2000 in the formulation may vary from 0 to 6% of the total formulation in the case of self-dispersible nanocarrier concentrate to be filled in capsules.

### Example 31: Self-Dispersible Cabazitaxel Oral Formulation

| | | Quantity | |
|---|---|---|---|
| S. No | Ingredients | (mg) | % w/v |
| 1 | cabazitaxel | 120 | 11.21 |
| 2 | cannabidiol | 50 | 4.67 |
| 3 | Labrafac Lipophile WL 1349 (Medium | 405 | 37.85 |
| | Chain Triglycerides NF) | | |
| 4 | decanoic acid | 50 | 4.67 |
| 5 | egg lecithin | 50 | 4.67 |
| 6 | N-(Carbonyl-methoxypolyethylenglycol- | 30 | 2.80 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Kolliphor RH40 | 100 | 9.35 |

### Manufacturing Procedure

Accurately weighed quantity of Labrafac Lipophile WL 1349 (Medium Chain Triglycerides NF) and Kolliphor RH40 were taken into a glass vial to which decanoic acid, PEG-PE, egg lecithin and cannabidiol were added and the contents were dissolved by heating on a water bath at 40° C. Then, required quantity of cabazitaxel was added to the above mixture followed by cooling to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 32: Self-Dispersible Cannabidiol Oral Liquid Oil

| | | Quantity | |
|---|---|---|---|
| S. No | Ingredients | (mg) | % w/v |
| 1 | cannabidiol synthetic | 8 | 0.75 |
| 2 | grapeseed oil | 400 | 37.38 |
| 3 | decanoic acid | 50 | 4.67 |
| 4 | egg lecithin | 50 | 4.67 |
| 5 | N-(Carbonyl-methoxypolyethylenglycol- | 0.3 | 0.03 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 6 | neotame | 30 | 2.80 |
| 7 | peppermint supreme | 20 | 1.87 |
| 8 | spearmint oil | 20 | 1.87 |
| 9 | cinnamon oil | 15 | 1.40 |
| 10 | menthol | 10 | 0.93 |
| 11 | ethanol | 100 | 9.35 |
| 12 | Kolliphor RH40 | 100 | 9.35 |
| 13 | Labrafac Lipophile WL 1349 (Medium | 197 | 18.41 |
| | Chain Triglycerides NF) | | |

### Manufacturing Procedure

Accurately weighed quantity of Labrafac Lipophile WL 1349 (Medium Chain Triglycerides NF), grapeseed oil and Kolliphor RH40 were taken into a glass vial to which decanoic acid, PEG-PE and egg lecithin were added and the contents were dissolved by heating on a water bath at 40° C. Then, required quantity of cannabidiol was added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

### Example 33: Self-Dispersible Cannabidiol Oral Liquid Oil

| | | Quantity | |
|---|---|---|---|
| S. No | Ingredients | (mg) | % w/v |
| 1 | cannabidiol synthetic | 8 | 0.75 |
| 2 | paclitaxel | 30 | 2.80 |
| 3 | grapeseed oil | 350 | 32.71 |
| 4 | decanoic acid | 50 | 4.67 |
| 5 | egg lecithin | 50 | 4.67 |
| 6 | N-(Carbonyl-methoxypolyethylenglycol- | 0.3 | 0.03 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 7 | neotame | 30 | 2.80 |
| 8 | peppermint supreme | 20 | 1.87 |
| 9 | spearmint oil | 20 | 1.87 |
| 10 | cinnamon oil | 15 | 1.40 |
| 11 | menthol | 10 | 0.93 |
| 12 | ethanol | 100 | 9.35 |
| 13 | Kolliphor RH40 | 100 | 9.35 |
| 14 | Labrafac Lipophile WL 1349 (Medium | 157 | 14.67 |
| | Chain Triglycerides NF) | | |

### Manufacturing Procedure

Accurately weighed quantity of Labrafac Lipophile WL 1349 (Medium Chain Triglycerides NF), grapeseed oil and Kolliphor RH40 were taken into a glass vial to which decanoic acid, PEG-PE and egg lecithin were added and the contents were dissolved by heating on a water bath at 40° C. Then, required quantity of cannabidiol followed by paclitaxel were added to the above mixture and dissolved by further heating at 40° C. to give a homogenous oil matrix which was then cooled to room temperature.

In another vial weighed quantity of ethanol was taken to which neotame, menthol, spearmint oil, cinnamon oil and peppermint supreme were added and dissolved by stirring for 5 min at 400 rpm. The ethanolic mixture of this vial was transferred to the vial containing cooled oil matrix and the resultant mixture was mixed for 2 min at 400 rpm.

The concentration of Lipoid PE 18:0/18:0-PEG 2000 in the formulation may vary from 5.61 to 18.69% (w/v) of the total formulation in the case of self-dispersible nanocarrier concentrate to be filled in capsules.

The concentration of Lipoid PE 18:0/18:0-PEG 2000 in the formulation may vary from 0.03 to 9.35% (w/v) of the total formulation in the case of orally administered dispersible liquid oil.

The sweeteners used in the case of orally administered dispersible liquid oil may be neotame, neohesperidine dihydrochalcone, sucralose, aspartame and erythritol.

The flavouring agents used may be single or a mixture of peppermint supreme, spearmint oil, cinnamon oil and menthol.

The surfactants used for improving the dispersibility of the formulations can be Kolliphor RH40, sucrose laurate, sucrose palmitate, Tween-80, Solutol HS15 or a combination of these surfactants.

### Example 34: Netupitant-CBD NLC Formulation (B #117A)

| | | Quantity | |
|---|---|---|---|
| S. No | Ingredients | (mg/mL) | %(w/v) |
| 1 | decanoic acid | 12 | 1.2 |
| 2 | Labrafac Lipophile WL 1349 (Medium | 12 | 1.2 |
| | Chain Triglycerides NF) | | |
| 3 | N-(Carbonyl | 3 | 0.3 |
| | methoxypolyethylenglycol-2000)-1,2- | | |
| | distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 4 | cannabidiol | 1.2 | 0.12 |
| 5 | netupitant | 4 | 0.4 |
| 6 | palonosetron hydrochloride | 0.005 | 0.0005 |
| 7 | ethanol | 10 | 1.0 |
| 8 | egg lecithin (Lipoid E80S) | 10 | 1.0 |
| 9 | cholesterol | 3 | 0.3 |
| 10 | poloxamer-188 | 10 | 1.0 |
| 11 | vitamin E acetate | 0.2 | 0.02 |
| 12 | ascorbyl palmitate | 0.2 | 0.02 |
| 13 | 1,4,7,10-Tetraazacyclododecane- | 0.02 | 0.002 |
| | 1,4,7,10-tetraacetic acid (DOTA) | | |
| 14 | chlorbutanol | 0.25 | 0.025 |
| 15 | L-histidine | 5 | 0.5 |
| 16 | dextrose anhydrous | 35 | 3.5 |
| 17 | Milli-Q Water | Q.S to | Q.S to |
| | | 1 mL | 100% |

### Manufacturing Procedure

Preparation of Oil Phase: Accurately weighed quantities of vitamin E acetate was taken into a manufacturing vessel. Labrafac Lipophile WL 1349 (oil) was subsequently added to it followed by decanoic acid and dissolved by heating on a water bath at 55° C. (50° C. to 60° C.). Weighed quantities of ascorbyl palmitate, cholesterol, PEG-PE and egg lecithin (Lipoid E80S) were further added and dissolved. This mixture was cooled to 40° C. and ethanol was added. Weighed amount of netupitant and CBD were added to the above mixture and dissolved to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose, chlorbutanol and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

Required quantity of Palonosetron HCl was added from prepared stock solution after high pressure homogenization. Finally, the pH of the preparation was adjusted in between 6.0-6.5 with L-histidine. The size and zeta potential of the undiluted NLC dispersion were measured using Particle Size Analyser (Lite Sizer, Anton Paar).

Further, the NLC formulation was subjected to long term at four temperature conditions (2-8° C., 25±2° C., 30±2° C. and 40±2° C.). The data evaluated in stability study is presented in Table 9 and Table 10.

TABLE 9
Particle size distribution data of netupitant-CBD NLC (B#117
A)

| | | | | | | | | Zeta |
|---|---|---|---|---|---|---|---|---|
| Stability | | HDD | PDI | D10 | D50 | D90 | SPA | Potential |
| Condition | | (nm) | (%) | (nm) | (nm) | (nm) | N | (mv) |
| Initial | | 196.24 | 21.83 | 106.90 | 191.67 | 320.49 | 1.12 | −16.20 |
| 6 | 2 − 8° C. | 207.73 | 19.03 | 124.15 | 196.95 | 299.73 | 0.89 | −19.93 |
| Months | 25 ± 2º C. | 225.95 | 18.70 | 129.51 | 219.97 | 358.13 | 1.03 | −9.53 |
| | 30 ± 2º C. | 234.95 | 17.50 | 141.56 | 222.24 | 340.54 | 0.89 | −12.37 |
| | 40 ± 2º C. | 207.56 | 18.90 | 123.33 | 193.26 | 302.59 | 0.93 | −6.30 |

TABLE 10
Assay and Impurity Profile of B#117A
Netupitant

| Batch No | | | 117A | | | |
|---|---|---|---|---|---|---|
| Condition | | Initial | 6M | | | |
| Time point | | | 2-8° C. | 25° C. | 30° C. | 40° C. |
| Name of the | RRT | | % w/w | | | |
| Impurity | | | | | | |
| Unknown | 0.8 | ND | ND | ND | ND | ND |
| Unknown | 0.91 | ND | ND | ND | ND | ND |
| Unknown | 0.95 | ND | 0.05 | 0.04 | 0.03 | 0.24 |
| Unknown | 1.05 | ND | ND | ND | ND | ND |
| N-Oxide | 1.08 | 0.1 | 0.09 | 0.5 | 0.59 | 0.32 |

| | | | | | |
|---|---|---|---|---|---|
| Total impurities | 0.1 | 0.14 | 0.54 | 0.62 | 0.56 |
| Assay | 105.2 | 103.7 | 101.9 | 102.1 | 99.8 |
| pH | 6.11 | 6.49 | 6.42 | 6.35 | 5.98 |
| Osmolality | NA | 313 | 309 | 313 | 310 |

Cannabidiol

| Condition | | Initial | 6M | | | |
|---|---|---|---|---|---|---|
| Time point | | | 2-8° C. | 25° C. | 30° C. | 40° C. |
| Related | RRT | % w/w | | | | |
| substances | | | | | | |
| Unknown | 2.54 | ND | ND | ND | ND | 0.02 |

| | | | | | |
|---|---|---|---|---|---|
| Total impurities | ND | ND | ND | ND | 0.02 |
| Assay | 105.2 | 105.3 | 102.4 | 103.2 | 96.6 |

Palonosetron

| Condition | Initial | 6M | | | |
|---|---|---|---|---|---|
| Time Point | | 2-8° C. | 25° C. | 30° C. | 40° C. |
| Related substances | % w/w | | | | |
| Unknown | ND | ND | ND | ND | ND |
| N-Oxide | ND | ND | ND | ND | 0.08 |
| Assay | 105.9 | 100.2 | 99.1 | 101.3 | 97.6 |

### Example 35: Cannabidiol (CBD) Oral NLC Formulation (B #130)

| | | Quantity | |
|---|---|---|---|
| S. No | Ingredients | (mg/mL) | % w/v) |
| 1 | decanoic acid | 8 | 0.8 |
| 2 | Labrafac Lipophile WL 1349 (Medium | 8 | 0.8 |
| | Chain Triglycerides NF) | | |
| 3 | N-(Carbonyl-methoxypolyethylenglycol- | 3 | 0.3 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 4 | cannabidiol synthetic | 8 | 0.8 |
| 5 | ethanol | 10 | 1 |
| 6 | egg lecithin (Lipoid E 80 S) | 5 | 0.5 |
| 7 | cholesterol | 3 | 0.3 |
| 8 | ascorbyl palmitate | 0.2 | 0.02 |
| 9 | propyl paraben | 0.2 | 0.02 |
| 10 | peppermint supreme | 1 | 0.1 |
| 11 | poloxamer-188 | 10 | 1 |
| 12 | DOTA | 0.02 | 0.002 |
| 13 | tris base | 1.5 | 0.15 |
| 14 | dextrose anhydrous | 35 | 3.5 |
| 15 | Milli-Q Water | Q.S to | Q.S to |
| | | 1 mL | 100% |

### Manufacturing Procedure

Preparation of Oil Phase: Accurately weighed quantity of Labrafac Lipophile WL 1349 (oil) was taken into a manufacturing vessel to which decanoic acid was added and dissolved by heating on a water bath at 55° C. (50° C. to 60° C.). Weighed quantities of cholesterol, PEG-PE and egg lecithin (Lipoid E80S) were subsequently added and dissolved to give a mixture which was cooled to 40° C. Weighed amount of CBD was added to the above mixture and dissolved. In another vessel peppermint supreme, propyl paraben, ascorbyl palmitate were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation was adjusted to 7.0-7.5 with Tris base. The size and zeta potential of the undiluted NLC dispersion was measured using particle size analyzer (Lite Sizer, Anton Paar).

Further, the NLC formulation was subjected to 1 year stability at 2-8° C. and 25±2° C. The data evaluated in stability study is presented in Table 11 and Table 12.

TABLE 11
Particle size distribution data of CBD Oral NLC (B#130)

| | | | | | | | | Zeta |
|---|---|---|---|---|---|---|---|---|
| | | | | | | | | Potent- |
| Stability | | HDD | PDI | D10 | D50 | D90 | | ial |
| Condition | | (nm) | (%) | (nm) | (nm) | (nm) | SPAN | (mv) |
| Initial | | 112.84 | 19.67 | 63.17 | 113.60 | 189.45 | 1.11 | −9.43 |
| 12M | 2-8° | 141.78 | 17.83 | 82.98 | 134.86 | 211.23 | 0.95 | −6.03 |
| | C. | | | | | | | |
| | 25 ± 2° | 154.13 | 17.57 | 94.12 | 143.02 | 216.07 | 0.85 | −9.03 |
| | C. | | | | | | | |

TABLE 12
Assay and impurity profile of cannabidiol oral NLC (B#130)
Cannabidiol

| Batch No | 130 | | |
|---|---|---|---|
| Condition | Initial | 12M | 12M |
| Time point | | 2-8° C. | 25° C. |
| Related substances | % w/w | | |
| Total impurities | ND | ND | ND |
| Assay | 101.7 | 98.3 | 96.6 |
| pH | 6.48 | 6.49 | 6.51 |
| Osmolality | NA | 266 | 271 |

*Note:
The formulation is Tetrahydrocannabinol (THC) free

Example 36
Cannabidiol (CBD) oral NLC formulation (B#013)

| | | Quantity | |
|---|---|---|---|
| S. No | Ingredients | (mg/mL) | %(w/v) |
| 1 | decanoic acid | 8 | 0.8 |
| 2 | Labrafac Lipophile WL 1349 | 8 | 0.8 |
| | (Medium Chain Triglycerides NF) | | |
| 3 | N-(Carbonyl- | 6 | 0.6 |
| | methoxypolyethylenglycol-2000)- | | |
| | 1,2-distearoyl-sn-glycero-3- | | |
| | phosphoethanolamine, MPEG- | | |
| | 2000-DSPE, Na-salt (Lipoid PE | | |
| | 18:0/18:0-PEG 2000) | | |
| 4 | cannabidiol synthetic | 8 | 0.8 |
| 5 | ethanol | 10 | 1.0 |
| 6 | egg lecithin (Lipoid E80S ) | 5 | 0.5 |
| 7 | cholesterol | 3 | 0.3 |
| 8 | poloxamer-188 | 10 | 1.0 |
| 9 | ascorbyl palmitate | 0.2 | 0.02 |
| 10 | 1,4,7,10-Tetraazacyclododecane- | 0.02 | 0.002 |
| | 1,4,7,10-tetraacetic acid (DOTA) | | |
| 11 | propyl paraben | 0.2 | 0.02 |
| 12 | tris base | 1.5 | 0.15 |
| 13 | dextrose anhydrous | 35 | 3.5 |
| 14 | neohesperidine dihydrochalcone | 0.5 | 0.05 |
| 15 | peppermint supreme | 1 | 0.1 |
| 16 | Milli-Q Water | Q.S to | Q.S to |
| | | 1 mL | 100% |

### Manufacturing Procedure

Preparation of Oil Phase: Accurately weighed quantity of Labrafac Lipophile WL 1349 (oil) was taken into a manufacturing vessel to which decanoic acid was added and dissolved by heating on a water bath at 55° C. (50° C. to 60° C.). Weighed quantities of cholesterol, PEG-PE and egg lecithin (Lipoid E80S) were subsequently added and dissolved to give a mixture which was cooled to 40° C. Weighed amount of CBD was added to the above mixture and dissolved. In another vessel peppermint supreme, propyl paraben, ascorbyl palmitate were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 and neohesperidine dihydrochalcone were dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation was adjusted to 7.0-7.5 with tris base. The size and zeta potential of the undiluted NLC dispersion was measured using particle size analyser (Lite Sizer, Anton Paar).

Further, the NLC formulation was subjected to stability at different temperature conditions. The data evaluated in stability study is presented in Table 13 and Table 14.

TABLE 13
Particle size distribution data of CBD oral NLC (B#013)

| | | | | | | | | Zeta |
|---|---|---|---|---|---|---|---|---|
| | | | | | | | | Potent- |
| Stability | | HDD | PDI | D10 | D50 | D90 | | ial |
| condition | | (nm) | (%) | (nm) | (nm) | (nm) | SPAN | (mv) |
| Initial | | 96.52 | 22.33 | 50.70 | 100.44 | 175.6 | 1.25 | −16.1 |
| 3 | 2-8° C. | 129.05 | 17.63 | 76.14 | 124.01 | 196.75 | 0.97 | −4.20 |
| Months | 25 ± 2° C. | 130.98 | 16.63 | 77.80 | 123.47 | 192.31 | 0.92 | −4.80 |

TABLE 14
Assay and impurity profile of cannabidiol oral NLC (B#013)
Cannabidiol

| Batch No | 013 | | |
|---|---|---|---|
| Condition | Initial | 3M | 3M |
| Time point | | 2-8° C. | 25° C. |
| Related | % w/w | | |
| substances | | | |
| Total impurities | ND | ND | 0.17 |
| Assay | 102 | 101.3 | 100.4 |
| pH | 6.5 | 6.64 | 6.64 |
| Osmolality | 242 | 218 | 212 |

*Note:
The formulation is Tetrahydrocannabinol (THC) free.

### Example 37: Cannabidiol (CBD) Oral NLC Formulation

| | | Quantity | |
|---|---|---|---|
| S. No | Ingredients | (mg/mL) | (% w/v) |
| 1 | Labrafac Lipophile WL 1349 (Medium | 10 | 1.0 |
| | Chain Triglycerides NF) | | |
| 2 | N-(Carbonyl-methoxypolyethylenglycol- | 6 | 0.6 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 3 | cannabidiol synthetic | 10 | 1.0 |
| 4 | ethanol | 10 | 1.0 |
| 5 | egg lecithin (Lipoid E80S ) | 5 | 0.5 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of Labrafac Lipophile WL 1349 (oil) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation was adjusted to 7.0-7.5 with Tris base. The size and zeta potential of the undiluted NLC dispersion was measured using particle size analyzer (Lite Sizer, Anton Paar).

TABLE 15
Particle size data of oral CBD NLC formulation

| | | | | | | Zeta |
|---|---|---|---|---|---|---|
| | | | | | | Potent- |
| HDD | | D10 | D50 | D90 | | ial |
| (nm) | PDI (%) | (nm) | (nm) | (nm) | SPAN | (mv) |
| 117.22 | 21.40 | 66.98 | 115.65 | 185.02 | 1.02 | −16.63 |

### Example 38: Cannabidiol (CBD) Oral NLC Formulation

| | | Quantity | |
|---|---|---|---|
| S. No | Ingredients | (mg/mL) | (% w/v) |
| 1 | Incromega E3322 (fish oil) | 10 | 1.0 |
| 2 | N-(Carbonyl- | 6 | 0.6 |
| | methoxypolyethylenglycol-2000)-1,2- | | |
| | distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 3 | cannabidiol synthetic | 10 | 1.0 |
| 4 | ethanol | 10 | 1.0 |
| 5 | egg lecithin (Lipoid E80S ) | 5 | 0.5 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of Incromega E3322 (Fish Oil) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation is adjusted to 7.0-7.5 with tris base.

### Example 39: Cannabidiol (CBD) Oral NLC Formulation

| | | Quantity | |
|---|---|---|---|
| S. No | Ingredients | (mg/mL) | (% w/v) |
| 1 | eicosapentaenoic acid (EPA) | 10 | 1.0 |
| 2 | N-(Carbonyl- | 6 | 0.6 |
| | methoxypolyethylenglycol-2000)- | | |
| | 1,2-distearoyl-sn-glycero-3- | | |
| | phosphoethanolamine, MPEG- | | |
| | 2000-DSPE, Na-salt (Lipoid PE | | |
| | 18:0/18:0-PEG 2000) | | |
| 3 | cannabidiol synthetic | 10 | 1.0 |
| 4 | ethanol | 10 | 1.0 |
| 5 | egg lecithin (Lipoid E80S ) | 5 | 0.5 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of eicosapentaenoic acid (EPA) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation is adjusted to 7.0-7.5 with tris base.

### Example 40: Cannabidiol (CBD) Oral NLC Formulation

| | | Quantity | |
|---|---|---|---|
| S. No | Ingredients | (mg/mL) | (% w/v) |
| 1 | docosahexaenoic acid (DHA) | 10 | 1.0 |
| 2 | N-(Carbonyl-methoxypolyethylenglycol- | 6 | 0.6 |
| | 2000)-1,2-distearoyl-sn-glycero-3- | | |
| | phosphoethanolamine, MPEG-2000- | | |
| | DSPE, Na-salt (Lipoid PE 18:0/18:0-PEG | | |
| | 2000) | | |
| 3 | cannabidiol synthetic | 10 | 1.0 |
| 4 | ethanol | 10 | 1.0 |
| 5 | egg lecithin (lipoid e80s ) | 5 | 0.5 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of docosahexaenoic acid (DHA) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation is adjusted to 7.0-7.5 with tris base.

### Example 41: Cannabidiol (CBD) Oral NLC Formulation

| | | Quantity | |
|---|---|---|---|
| S. No | Ingredients | (mg/mL) | (% w/v) |
| 1 | Labrafac Lipophile WL 1349 (Medium | 15 | 1.5 |
| | Chain Triglycerides NF) | | |
| 2 | N-(Carbonyl-methoxypolyethylenglycol- | 6 | 0.6 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG2000) | | |
| 3 | cannabidiol synthetic | 15 | 1.5 |
| 4 | ethanol | 10 | 1.0 |
| 5 | egg lecithin (Lipoid E80S ) | 10 | 1.0 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of Labrafac

Lipophile WL 1349 (oil) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation is adjusted to 7.0-7.5 with tris base.

### Example 42: Cannabidiol (CBD) Oral NLC Formulation

| | | Quantity | |
|---|---|---|---|
| S. No | Ingredients | (mg/mL) | (% w/v) |
| 1 | Labrafac Lipophile WL 1349 (Medium | 20 | 2.0 |
| | Chain Triglycerides NF) | | |
| 2 | N-(Carbonyl-methoxypolyethylenglycol- | 6 | 0.6 |
| | 2000)-1,2-distearoyl-sn-glycero-3- | | |
| | phosphoethanolamine, MPEG-2000- | | |
| | DSPE, Na-salt (Lipoid PE 18:0/18:0- | | |
| | PEG 2000) | | |
| 3 | cannabidiol synthetic | 20 | 2.0 |
| 4 | ethanol | 10 | 1.0 |
| 5 | egg lecithin (Lipoid E80S ) | 10 | 1.0 |
| 6 | poloxamer-188 | 10 | 1.0 |
| 7 | ascorbyl palmitate | 0.2 | 0.02 |
| 8 | 1,4,7,10-Tetraazacyclododecane- | 0.02 | 0.002 |
| | 1,4,7,10-tetraacetic acid (DOTA) | | |
| 9 | propyl paraben | 0.2 | 0.02 |
| 10 | tris base | 0.1 | 0.01 |
| 11 | dextrose anhydrous | 35 | 3.5 |
| 12 | neotame | 1 | 0.1 |
| 13 | peppermint supreme | 2 | 0.2 |
| 14 | menthol | 0.4 | 0.04 |
| 15 | Milli-Q Water | Q.S to | Q.S to |
| | | 1 mL | 100% |

### Manufacturing Procedure

Preparation of Oil Phase: Accurately weighed quantity of Labrafac Lipophile WL 1349 (oil) was taken into a manufacturing vessel. Weighed quantities of PEG-PE and egg lecithin (Lipoid E80S) were subsequently added and dissolved by heating on a water bath at 55° C. (50° C. to 60° C.) to give a mixture which was cooled to 40° C. Weighed amount of CBD was added to the above mixture and dissolved. In another vessel peppermint supreme, propyl paraben, ascorbyl palmitate, neotame and menthol were dissolved in ethanol. The contents of both the vessels were mixed to give a lipid phase.

Preparation of Aqueous Phase: 90% of required quantity of Milli-Q water per batch was taken in another manufacturing vessel in which weighed quantity of poloxamer-188 was dissolved by heating at 55° C. (50° C. to 60° C.). Dextrose and DOTA were subsequently added and dissolved in the above solution.

### Preparation of NLC

High Shear Homogenization: The lipid phase was added into the aqueous phase, under homogenization at 4000 rpm by maintaining the product temperature at 55° C. (50° C. to 60° C.) and the homogenization was continued further for 15 min at 8500 rpm. Later, the volume was adjusted up to required level with remaining quantity of Milli-Q water followed by homogenization at 8500 rpm for 15 minutes for formation of a coarse dispersion.

High Pressure Homogenization: The obtained coarse dispersion was subjected to high pressure homogenization for 3 passes at different pressures i.e. Pass 1 at 5,000 psi, Pass 2 at 10,000 psi and Pass 3 at 18,000 psi.

The pH of the formulation is adjusted to 7.0-7.5 with tris base.

1. An oral nanolipid carrier system comprising:
i. one or more low phase transition substances selected from the group comprising a low phase transition lipid, cannabidiol or mixtures thereof,
ii. oil,
iii. PEGylated lipid,
iv. surfactant, and
v. a co-solvent.

2. The carrier system according to claim 1, wherein the cannabidiol permeation rate is increased by about 2 fold.

3. The carrier system according to claim 1, further comprising a hydrodynamic diameter which when measured in aqueous dispersion is less than about 1000 nm.

4. The carrier system according to claim 3, wherein the hydrodynamic diameter when measured in aqueous dispersion is less than about 700 nm.

5. The carrier system according to claim 1, wherein PEG is attached to the PEGylated lipid and the PEG has a molecular weight which ranges from 200Da to 20000 Da.

6. The carrier system according to claim 1, wherein the PEGylated lipid is PEG-PE.

7. The carrier system according to claim 1, wherein the low phase transition lipid used has a phase transition temperature below 80° C.

8. The carrier system according to claim 1, wherein the low phase transition lipid is present up to 20% by weight of the formulation.

9. The carrier system according to claim 1, wherein cannabidiol is present up to 35% by weight of the composition.

10. The carrier system according to claim 1, wherein the oil is selected from the group comprising medium chain triglycerides, fish Oil, grape seed oil and soyabean oil.

11. The carrier system according to claim 1, wherein the low phase transition lipid is selected from the group comprising: decanoic acid, Monosteol, alpha lipoic acid and cholesterol.

12. The carrier system according to claim 1, wherein the low phase transition substance is cannabidiol.

13. An oral nanolipid drug-carrier system, comprising:
i. cannabidiol
ii. oil,
iii. PEG-PE,
iv. surfactant, and
v. a co-solvent.

14. The carrier system according to claim 13, wherein the cannabidiol permeation rate is increased by about 2 fold.

15. An oral nanolipid carrier system comprising:
i. Cannabidiol,
ii. One or more active ingredients
iii. oil,
iv. PEGylated lipid,
vv. surfactant, and
vi. a co-solvent.

16. The carrier system according to claim 15 where in the one or more active ingredients are selected from the group comprising netupitant, palonosetron, atorvastatin, gabapentin, naproxen, abiraterone, cabazitaxel, paclitaxel, duloxetine and many other drugs and there combinations thereof.

17. The carrier system according to claim 15, wherein the active ingredient permeation rate is increased by about 2 fold.
